(19) United States
(12) Patent Application Publication
(10) Pub. No.: US 2017/0224217 A1
(43) Pub. Date: Aug. 10, 2017

(54) SYSTEMS AND METHODS FOR MAGNETIC RESONANCE BLACK-BLOOD THROMBUS IMAGING IN DETECTION OF CEREBRAL VENOUS THROMBOSIS

(71) Applicant: Cedars-Sinai Medical Center, Los Angeles, CA (US)

(72) Inventors: Qi Yang, Los Angeles, CA (US); Zhaoyang Fan, Hacienda Heights, CA (US); Debiao Li, So. Pasadena, CA (US); Xunming Ji, Beijing (CN)

(73) Assignee: Cedars-Sinai Medical Center, Los Angeles, CA (US)

(21) Appl. No.: 15/015,904

(22) Filed: Feb. 4, 2016

(51) Int. Cl. A61B 5/00; A61B 5/055

(52) U.S. Cl. CPC A61B 5/0042; A61B 5/055; A61B 5/4064; A61B 2576/026

(57) ABSTRACT

In various embodiments, the present invention teaches systems and methods for using T1-weighted black-blood MR imaging, with which a CVT can be well isolated from the surrounding tissues due to the signal suppression of flowing blood. In some embodiments, the invention teaches using black-blood imaging (3D variable-flip-angle turbo spin-echo acquisition) to directly visualize thrombi. In certain embodiments, the invention teaches using T1 weighted image contrast and isotropic sub-millimeter spatial resolution for accurate detection and staging of thrombi. In various embodiments, the invention allows for the detection of chronic thrombosis recanalization.

### FIELD OF THE INVENTION

The present invention generally relates to imaging methods, and especially magnetic resonance imaging methods.

### BACKGROUND

The following description includes information that may be useful in understanding the present invention. It is not an admission that any of the information provided herein is prior art or relevant to the presently claimed invention.

Cerebral venous thrombosis (CVT), including thrombosis of cerebral veins and major dural sinuses, is a form of stroke that usually affects young individuals. During the past decade, improved diagnosis and treatments have improved the outcome of CVT. However, CVT is frequently unrecognized and the average delay from the onset of symptoms to the diagnosis is about one week, because the positive findings of intraluminal thrombus are not always evident. Diagnosis of CVT typically relies on a combination of different imaging modalities, such as computed tomography (CT) venography, magnetic resonance (MR) venography, and conventional X-ray angiography. These methods assess CVT indirectly by imaging venous flow perturbation caused by thrombus. However, given the variation in venous anatomy, it is sometimes difficult to exclude CVT with existing noninvasive imaging modalities. The diagnosis dilemma may delay treatment and result in death or permanent disability. There are still many unsolved issues in the pathophysiology, diagnosis, and management of CVT. New system and methods for magnetic resonance imaging (MM) could increase the accuracy of diagnosis of CVT.

One general solution to these limitations is the direct visualization of the thrombus itself. Magnetic resonance direct thrombus imaging (MRDTI), as a non-contrast-enhanced T1-weighted imaging method, has gained broad interest. Several studies have confirmed a high sensitivity of detecting thrombus in the coronary artery, carotid artery, deep vein, and pulmonary artery. By exploiting the short T1 relaxation time of methemoglobin within thrombus, MRDTI depicts subacute thrombus as hyper-intense signal while maintaining background tissues such as the blood, vessel wall, and surrounding brain tissues as isointense signal. However, the signal intensity of evolving thrombus may be complicated by coexisting more acute and older thrombus components which may appear isointense as well. As a result, part of a thrombus could be mistaken as venous blood or surrounding brain tissues. It was thought to be even more challenging when utilizing MRDTI in the cerebral venous system where anatomic variants including sinus atresia/hypoplasia asymmetrical sinus drainage are commonly present.

There is a need in the art for improved systems and methods for detecting CVT through MRDTI.

### SUMMARY OF THE INVENTION

In various embodiments, the invention teaches a method for performing magnetic resonance imaging (MM) on a subject, including (1) acquiring magnetic resonance data from a volume of interest (VOI) that includes a blood vessel of a subject's head and/or neck, by using a 3D variable-flip-angle turbo spin-echo (TSE) acquisition, and (2) generating one or more images based on said data. In some embodiments, the blood vessel includes a thrombus. In certain embodiments, the thrombus is depicted as hyperintense compared with surrounding tissues. In some embodiments, the subject has cerebral venous thrombosis (CVT). In some embodiments, the method further includes using T1-weighting. In some embodiments, the MRI machine used to perform the magnetic resonance imaging is a 1.5 T scanner or a 3.0 T scanner. In various embodiments, the VOI includes one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In some embodiments, the subject is a human.

In various embodiments, the invention teaches a magnetic resonance imaging (MRI) system that includes (1) a magnet operable to provide a magnetic field; (2) a transmitter operable to transmit to a region within the magnetic field; (3) a receiver operable to receive a magnetic resonance signal from the region; and (4) a processor operable to control the transmitter and the receiver; wherein the processor is configured to direct the transmitter and receiver to execute a sequence that includes (a) acquiring magnetic resonance data from a blood vessel within a volume of interest (VOI) that includes all or a portion of a subject's head and/or neck, according to the method described above, and (b) generating one or more images based on the magnetic resonance data acquired. In some embodiments, the system includes a head and/or neck coil. In certain embodiments, the system is configured to image one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In some embodiments, the subject is a human. In certain embodiments, the system includes a subsystem configured to accelerate imaging speed via parallel processing. In certain embodiments, the MRI system is a 1.5 T system or a 3.0 T system.

In various embodiments, the invention teaches a non-transitory machine-readable medium having machine executable instructions for causing one or more processors of a magnetic resonance imaging (MRI) machine, and/or a subsystem configured to function therewith, to execute an imaging method, said method including: performing a 3D variable-flip-angle turbo spin-echo (TSE) acquisition of a blood vessel within a volume of interest (VOI) that includes a subject's head and/or neck. In certain embodiments, the imaging includes T1-weighting. In certain embodiments, the MRI machine includes a head and/or neck coil. In certain embodiments, the VOI includes one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In some embodiments, the blood vessel includes a thrombus. In certain embodiments, the subject is a human.

### BRIEF DESCRIPTION OF THE DRAWINGS

Exemplary embodiments are illustrated in the referenced figures. It is intended that the embodiments and figures disclosed herein are to be considered illustrative rather than restrictive.

FIGS. 1A-1D depict, in accordance with an embodiment of the invention, magnetic resonance black-blood thrombus imaging (MRBTI) images of a 48-year-old male with subacute cerebral venous thrombosis. On time-of-flight (TOF) images, normal venous sinuses were depicted with bright venous flow signals (arrowheads in A and B), and minor flow defects (black arrowhead in A) observed in the superior sagittal sinus were not considered as thrombus by radiologists. With blood signal adequately suppressed on MRBTI, normal venous sinus were depicted as black area (arrowheads in C and D), and hyperintense signal was found in the right transverse sinus suggesting a fresh thrombus (arrow in D). The thrombus was also confirmed on TOF (arrow in B).

FIG. 2 depicts, in accordance with an embodiment of the invention, high thrombus signal/noise ratio (SNR), thrombus-to lumen contrast/noise ratio (CNR), and thrombus-to-gray matter CNR were obtained with magnetic resonance black-blood thrombus imaging in both thrombus groups. They were all significantly different between the 2 groups.

FIGS. 3A-3C demonstrate, in accordance with an embodiment of the invention, magnetic resonance black-blood thrombus imaging (MRBTI) of a 27-year-old male patient with subacute cerebral venous thrombosis. FIG. 3A, MRBTI demonstrated hyperintense signal intensity in the superior sagittal sinus (arrowheads), the right transverse and sigmoid sinuses (arrowheads), and the cortical veins (arrows) suggesting intraluminal thrombus formation. FIG. 3B, All thrombi semiautomatically outlined by software based on their high signal contrast were rendered with red color, and volume was 21.5 mL. FIG. C, Sagittal, coronal, and axial sections of maximum intensity projection reformations of MRBTI better depicted the thrombosed segments with hyperintense signals.

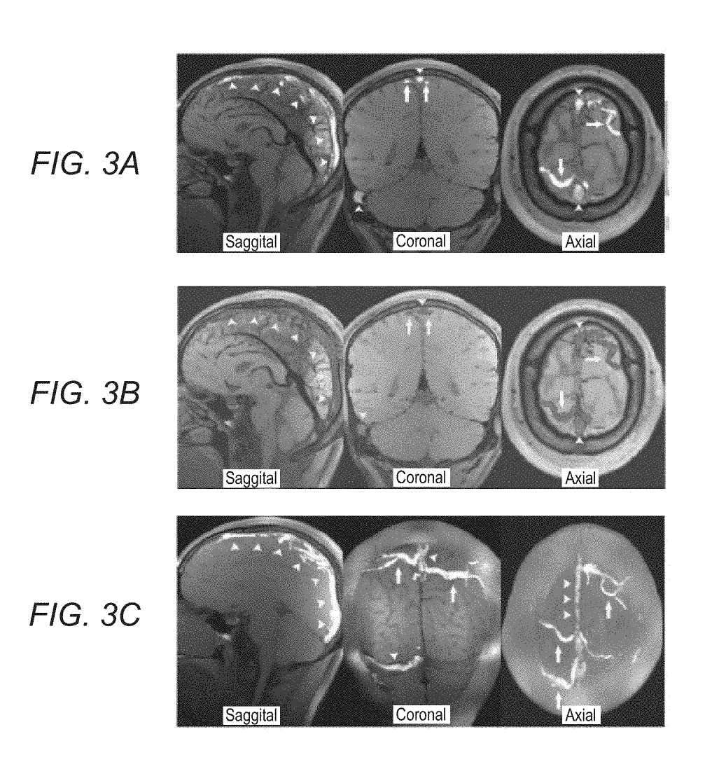

FIGS. 4A-4D demonstrate, in accordance with an embodiment of the invention, magnetic resonance black-blood thrombus imaging (MRBTI) of a 23-year-old female patient who was imaged on day 7 (A and B) and day 14 (C and D) after symptomatic onset. MRBTI demonstrated hyperintense signal intensity in left transverse sinus suggesting intraluminal thrombus formation. The thrombus on day 7 (arrowheads) and day 14 (arrows) exhibited different hyperintense signal patterns. Cerebral venous thrombosis volume was 22.4 and 12.5 mL on day 7 and day 14, respectively. The right transverse sinus with suppressed lumen signals became larger in size in response to the gradual occlusion of the left transverse sinus.

FIG. 5 depicts a system in accordance with an embodiment of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Westbrook et al., MRI in Practice 4^{th} ed., and Guyton and Hall, Textbook of Medical Physiology 12^{th} ed., provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, certain terms are defined below.

“Conditions” and “disease conditions,” as used herein, may include but are in no way limited to thrombosis, including but in no way limited to cerebral venous thrombosis (CVT), intracoronary thrombus, deep vein thrombosis, and pulmonary emboli.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult and newborn subjects, whether male or female, are intended to be included within the scope of this term.

Importantly, 3D variable-flip-angle TSE has an inherent flow dephasing effect that can be utilized to null the signal from flowing blood. This “black-blood” effect may be further improved by using additional black-blood preparation, such as DANTE (Delay alternating with nutation for tailored excitation) preparation that consists of a train of hard RF pulses interspersed with dephasing gradients (See Li L, Miller K L, Jezzard P. DANTE-prepared pulse trains: a novel approach to motion-sensitized and motion-suppressed quantitative magnetic resonance imaging. Magn Reson Med 2012; 68: 1423-1438, which is hereby incorporated herein by reference in its entirety as though fully set forth). Black-blood thrombus imaging (BTI) allows CVT to be well isolated from the surrounding tissues including luminal blood flow and the vessel wall, and thus the location and size of CVT can be readily appreciated. Additionally, BTI enables quantification of thrombus volume. Due to the high signal contrast of thrombus and high isotropic spatial depiction of venous structure, the volume of CVT can be quantified in a semiautomatic or even automatic fashion (e.g., by utilizing a computing device to ascertain dimensions based on the image(s)). This is especially useful for monitoring thrombus progression and evaluating treatment response. In addition, normal sinus anatomy or structure variants, such as arachnoid granulations and hypoplasia, can be well-visualized with BTI, which can be valuable for interventional planning.

With the foregoing background in mind, in various embodiments the present invention teaches methods using T1-weighted black-blood magnetic resonance (MR) imaging, with which a CVT can be well isolated from the surrounding tissues due to the signal suppression of flowing blood. In some embodiments, the invention teaches using black-blood imaging (3D variable-flip-angle turbo spin-echo acquisition) to directly visualize thrombi. In certain embodiments, the invention teaches using T1-weighted image contrast and isotropic sub-millimeter spatial resolution for accurate detection and staging of thrombi. In various embodiments, the invention allows for the detection of chronic thrombosis recanalization. In some embodiments, the invention teaches the use of a head/neck combined coil, with which comprehensive assessment of cerebral venous thrombosis can be performed with a large spatial coverage from the skull to the neck.

In various embodiments, the invention teaches a method for performing magnetic resonance imaging (MRI) on a subject. In some embodiments, the method includes (1) acquiring magnetic resonance data from a volume of interest (VOI) that includes a blood vessel of the subject's head and/or neck, by using an MRI machine to perform a 3D variable-flip-angle turbo spin-echo (TSE) acquisition, and (2) generating one or more images based on said data. In certain embodiments, the blood vessel includes a thrombus. In some embodiments, imaging parameters include oblique coronal single-slab coverage, repetition time=500-1000 ms, echo time=5-25 ms, matrix=160×160 to 320×320, field of view=200×200 to 400×400 mm2, slice thickness=0.5-1.5 mm, slices=50-250, and scan time=3-8 minutes. In some embodiments, imaging parameters include oblique coronal single-slab coverage, repetition time=800 ms, echo time=22 ms, matrix=198×192, field of view=160×200 mm2, slice thickness=0.6-1.0 mm, slices=100-200, and scan time=6-8 minutes. In some embodiments, the thrombus is depicted as hyperintense compared with surrounding tissues, as shown in the referenced figures. In certain embodiments, the subject has cerebral venous thrombosis (CVT). In some embodiments, T1 weighted image contrast is used. In some embodiments, the MRI machine used in conjunction with the inventive methods described herein is a 1.5 T scanner or a 3.0 T scanner. One of skill in the art would readily appreciate that a scanner of any appropriate strength could be utilized in conjunction with the inventive methods. In certain embodiments, the VOI includes, but is in no way limited to one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In certain embodiments, the subject is a mammal. In some embodiments, the subject is a human.

In various embodiments, the invention teaches a magnetic resonance imaging (MRI) system, that includes (1) a magnet operable to provide a magnetic field; (2) a transmitter operable to transmit to a region within the magnetic field; (3) a receiver operable to receive a magnetic resonance signal from the region; and (4) a processor operable to control the transmitter and the receiver. In some embodiments, the processor is configured to direct the transmitter and receiver to execute a sequence that includes (a) acquiring magnetic resonance data from a blood vessel within a volume of interest (VOI) which includes all or a portion of a subject's head and/or neck, according to the methods described herein, and (b) generating one or more images based on the magnetic resonance data acquired. In certain embodiments the imaging parameters are within the range of imaging parameters described herein. In certain embodiments, the system includes a head and/or neck coil. In some embodiments, the system is configured to image one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In some embodiments, the subject is a human. In some embodiments, the MM system includes a subsystem configured to accelerate imaging speed via parallel processing. In certain embodiments, the MM system is a 1.5 T system or a 3.0 T system, but one of skill in the art would readily appreciate that an MRI system of any appropriate strength could be used.

In various embodiments, the invention teaches a non-transitory machine-readable medium having machine executable instructions for causing one or more processors of a magnetic resonance imaging (MRI) machine, and/or a subsystem configured to function therewith, to execute an imaging method, said method including: performing a 3D variable-flip-angle turbo spin-echo (TSE) acquisition of a blood vessel within a volume of interest (VOI) that includes a subject's head and/or neck. In some embodiments, the imaging includes T1-weighting. In various embodiments of the invention, the MRI machine used in conjunction with the non-transitory machine-readable medium includes a head and/or neck coil. In some embodiments, the VOI includes one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins. In certain embodiments, the imaging parameters are within the range of imaging parameters described herein. In some embodiments, the blood vessel imaged includes a thrombus. In some embodiments, the subject is a human.

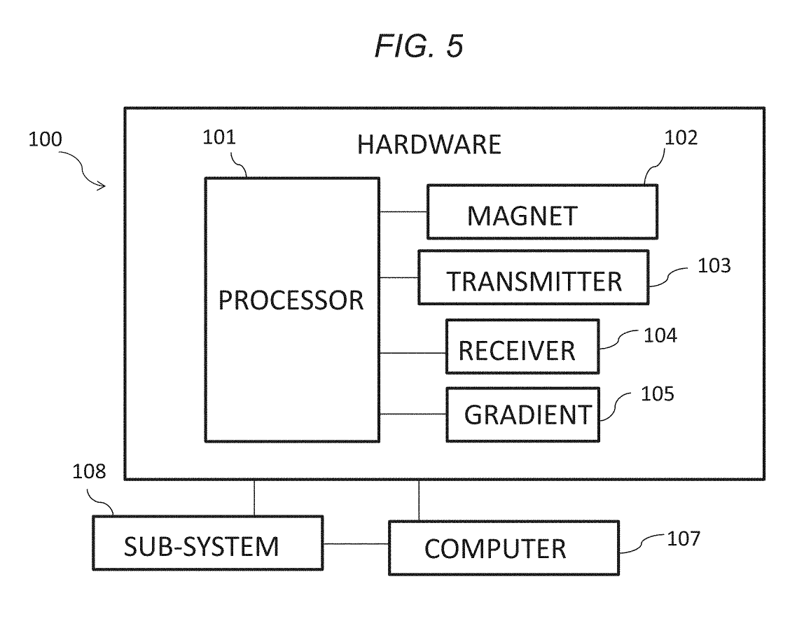

One of skill in the art would readily appreciate that several different types of imaging systems could be used to perform the inventive methods described herein. Merely by way of example, the imaging systems described in the examples could be used. FIG. 5 also depicts a view of a system 100 that can be used to accomplish the inventive methods. System 100 includes hardware 106 and computer 107. Hardware 106 includes magnet 102, transmitter 103, receiver 104, and gradient 105, all of which are in communication with processor 101. Magnet 102 can include a permanent magnet, a superconducting magnet, or other type of magnet. Transmitter 103 along with receiver 104, are part of the RF system. Transmitter 103 can represent a radio frequency transmitter, a power amplifier, and an antenna (or coil). Receiver 104, as denoted in the figure, can represent a receiver antenna (or coil) and an amplifier. In the example shown, transmitter 103 and receiver 104 are separately represented, however, in one example, transmitter 103 and receiver 104 can share a common coil. Hardware 106 includes gradient 105. Gradient 105 can represent one or more coils used to apply a gradient for localization. In some embodiments, the receiver coil set up can be achieved either by combining commercial head and neck coils or by designing a new head/neck coil.

Processor 101, in communication with various elements of hardware 106, includes one or more processors configured to implement a set of instructions corresponding to any of the methods disclosed herein. Processor 101 can be configured to implement a set of instructions (stored in memory of hardware 106 or sub-system 108 or otherwise accessible through an alternative source) to provide RF excitation and gradients and receive magnetic resonance data from a volume of interest. Sub-system 108 can include hardware and software capable of facilitating the processing of data generated by hardware 106, in conjunction with, or as a substitute for, the processing associated with image reconstruction that is normally handled by processor 101 in an MRI machine. One of skill in the art would readily appreciate that certain components of the imaging systems described herein, including the processor 101 and/or sub-system 108, are used to execute instructions embedded on a computer readable medium to implement the inventive data acquisition and image reconstruction methods described herein.

In some embodiments, computer 107 is operably coupled to hardware 106 and sub-system 108. Computer 107 can include one or more of a desktop computer, a workstation, a server, or a laptop computer. In one example, computer 107 is user-operable and includes a display, a printer, a network interface or other hardware to enable an operator to control operation of the system 100.

In some embodiments, the invention includes using any of the methods or systems described herein to diagnose a subject with the presence or absence of a thrombus and/or size and/or location and/or age of a thrombus, based upon the data and/or images acquired. In some embodiments, the invention includes using any of the methods or systems described herein to diagnose a subject with the presence or absence of CVT, based upon the data and/or images acquired. In some ernbodimnents, the size and/or age and/or position of the thrombus is determined, manually or automatically, based on the imaging data and/or one or more image resulting from the methods described herein.

In some embodiments, the invention includes treating a patient who was diagnosed with CVT (or any other type of thrombus) after imaging with BTI according to any method described herein. In some embodiments, the treatment may include administering a therapeutic amount of a thrombolytic drug, an anticoagulant drug, or a related therapeutic. In some embodiments, the treatment may include, but is in no way limited to, administering low-molecular-weight heparin or dose-adjusted intravenous heparin (or an alternative drug with a similar effect). In some embodiments, antithrombotic treatment with urokinase (or an alternative drug with a similar effect) may be used to recanalise the occluded sinus or vein, to prevent the propagation of the thrombus, and to prevent venous thrombosis in other parts of the body. In some embodiments, the treatment may also include, or may alternatively include, a surgical intervention of a type such as, but is in no way limited to, balloon-assisted thrombectomy and/or thrombolysis, catheter thrombectomy, and the like, and combinations thereof. Any other standard treatments may also be administered to the subject diagnosed as having a thrombus without departing from the spirit of the invention

One skilled in the art will recognize many methods and materials similar or equivalent to those described herein, which could be used in the practice of the present invention. Indeed, the present invention is in no way limited to the methods and materials described.

### EXAMPLES

### Example 1

### Subjects and Methods

### Patients

Between February 2014 and May 2015, consecutive patients with signs and symptoms suspected of CVT within the past 30 days were prospectively recruited. Exclusion criteria included general contraindications to MR examination and patients with incomplete conventional imaging examinations (CT, MR, and MRV). Informed consent was obtained from all participants, and all protocols were approved by the Institutional Review Board.

### Conventional Imaging Evaluation

Thrombi were defined as intraluminal filling defects detected by conventional imaging techniques. Two readers (J.D. and X.J.) performed a consensus reading of all conventional imaging studies for each patient, including CT, MR, and MRV, with full clinical and outcome information on the patient to obtain a reference standard. The following 14 venous segments were included in evaluations: superior sagittal sinus, inferior sagittal sinus, right transverse sinus, right sigmoid sinus, left transverse sinus, left sigmoid sinus, straight sinus, confluence of sinuses, veins of galen, internal cerebral veins, basal veins of rosenthal, veins of Labbé, right cortical veins, and left cortical veins.

### Magnetic Resonance Black-Blood Thrombus Imaging

As indicated above, 3D variable flip angle turbo spin echo has an inherent black-blood effect and has recently been proposed for arterial vessel wall imaging (See Mugler J P 3^{rd}. Optimized three-dimensional fast-spin-echo MRI. J Magn Reson Imaging. 2014; 39:745-767, which is hereby incorporated herein by reference in its entirety as though fully set forth). To exploit the short T1 property of acute thrombus, the sequence was used for MRBTI with a T1-weighted acquisition mode. All MR studies were conducted on a 3.0-T system (MAGNETOM Verio, Siemens Healthcare, Erlangen, Germany) using a 32-channel head coil for signal reception. Typical imaging parameters included oblique coronal single-slab coverage, repetition time=800 ms, echo time=22 ms, matrix=198×192, field of view=160×200 mm2, slice thickness=0.6-1.0 mm, slices=100-200, and scan time=6-8 minutes.

### MRBTI Image Evaluation

All MRBTI images were randomized and presented to 2 independent readers with 10 years (Q.Y.) and 8 years (X.Q.) of experience in reading. The readers were not involved with the diagnostic or therapeutic management of the patients and were blinded to clinical information and conventional imaging data on which the diagnosis of CVT was based. Source images, free mode multiplanar reformation, and minimum intensity projection images were used by readers. A third reader with 15 years of experience of reading (K.L.) was involved to resolve any disputes.

Image quality was first rated for each segment using a 4-point scale as follows: 4=excellent, no relevant artifacts; 3=good, minimal inhomogeneity, only minor flow artifacts; 2=adequate, delineated lumen, major flow artifacts; and 1=nondiagnostic. Thrombus was visually assessed in each segment based on its characteristic hyperintense signals relative to the luminal blood and surrounding brain tissues. The presence or absence of thrombus was recorded for each segment. Patients with CVT detected by MRBTI were divided into 2 groups based on the duration of clinical onset: ≦7 days (group 1) and between 7 and 30 days (group 2). Signal intensity was measured from thrombus, luminal blood, and gray matter. Signal/noise ratio was calculated for the detected thrombus and was defined as the ratio of the thrombus signal intensity and SD of the background noise measured in an area outside of the head free of tissue structure and artifact. Contrast/noise ratio (CNR) was measured between thrombus and lumen and also between thrombus and gray matter. CNR was calculated as signal intensity difference between the thrombus and lumen/gray matter divided by the SD of background noise. In addition, the feasibility of using MRBTI for thrombus volume measurement was explored. Specifically, thrombi in each patient were segmented in a semiautomatic fashion using commercial software (Object Research System, Montreal, Quebec, Canada), and total thrombus volume was reported for each patient.

### Statistical Analysis

Differences in signal/noise ratio and CNRs between group 1 and group 2 were tested with 2-tailed independent t-test. A value of P<0.05 was considered to indicate statistical significance. The level of agreement in thrombus detection between the 2 readers was evaluated by the κ value on a per-segment basis. The consensus reading of conventional imaging techniques was used as the reference standard for assessing the sensitivity, specificity, and negative and positive predictive values of MRBTI. For the patient level analysis, each patient was categorized as correctly diagnosed if at least 1 venous segment was judged as positive CVT. All statistical analysis was performed using statistical software (SAS version 9.1, SAS Institute Inc, Cary, N.C.).

### Results

### Patients Characteristics

Sixty-two consecutive patients met the eligibility criteria, and 15 patients were excluded because of incomplete imaging at baseline. Thus, 47 patients were enrolled in the MRBTI examination. MRBTI was successfully performed in all 47 patients without complications. The mean age of the patients in the study was 34 years (range, 5-84 years), and 28 (60%) were women. Study population characteristics are listed in Table 1.

TABLE 1
Baseline Study Population Characteristics

| | n/N (%) | |
|---|---|---|
| Demographics | | |
| Mean age, y (SD) | 34 ± 13 | |
| Sex, female (%) | 28/47 | (60) |
| Clinical characteristics (%) | | |
| Headache | 27/47 | (57%) |
| Papilledema | 12/47 | (26) |
| Focal neurological deficit | 5/47 | (11) |
| Comatose | 1/47 | (2) |
| Duration from onset to MRBTI, d(%) | | |
| 0-7 | 19/47 | (40) |
| 7-30 | 28/47 | (60) |
| Risk factors | | |
| Pregnancy or puerperium (% of women) | 6/47 | (13) |
| Oral contraceptives (% of women) | 8/47 | (17) |
| Infection (%) | 7/47 | (15) |

MRBTI indicates magnetic resonance black-blood thrombus imaging

### Distribution of Venous Thrombosis by Conventional Imaging Techniques

All 47 patients have CT, MR, and time-of-flight MRV; 5 of 47 patients have contrast-enhanced CT venography. A total of 116 thrombosed venous segments were identified in 23 patients. Thrombosed segments included superior sagittal sinus (14), right transverse sinus (16), right sigmoid sinus (17), left transverse sinus (11), left sigmoid sinus (9), straight sinus (8), confluence of sinuses (12), veins of galen (4), internal cerebral veins (2), veins of Labbé (1), right cortical veins (11), and left cortical veins (11).

### MRBTI Image Quality

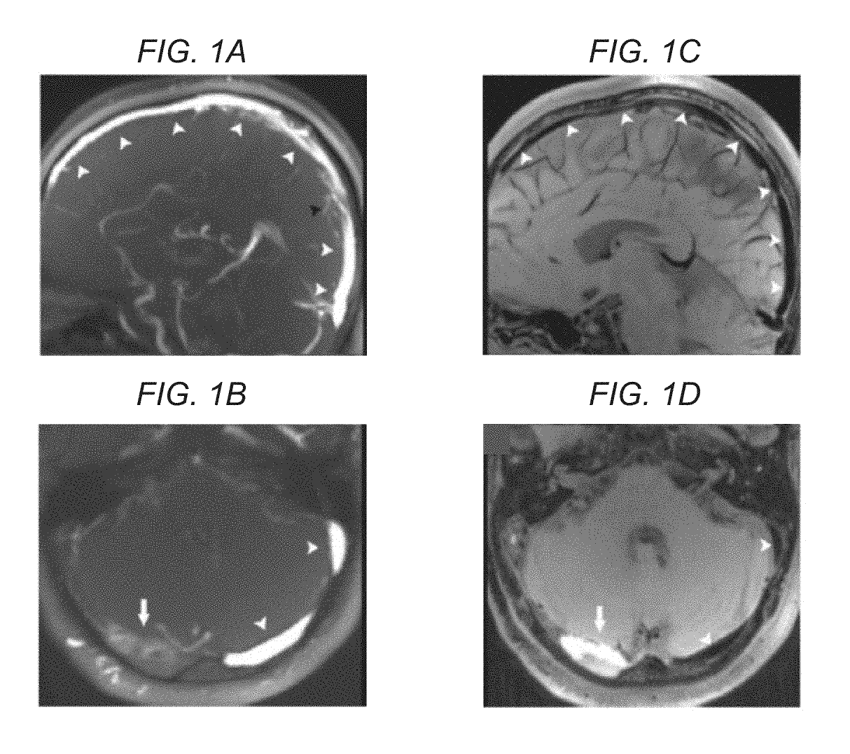

FIG. 1 shows a typical example of a thrombosis case acquired with a conventional time-of-flight technique and the MRBTI method. Blood signal was effectively suppressed using MRBTI, and thrombi were depicted as hyperintense with excellent contrast relative to surrounding tissues (FIGS. 1B and 1D). In comparison, some flow dephasing-related signal loss was observed in time-of-flight images (FIG. 1A, arrowheads). The overall image quality score was 3.5±0.6. Among 658 segments, 647 (98%) were diagnostic (score,≧2). Among the nondiagnostic segments, 7 were due to limited spatial coverage and 4 were due to flow artifacts. These 11 segments were excluded in the following diagnostic performance analyses.

### Thrombus Signal Intensity

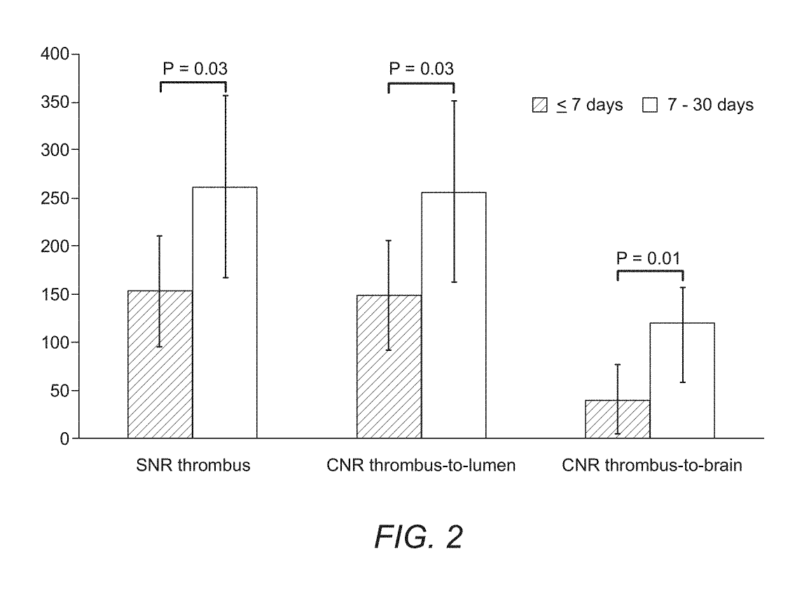

All thrombi were depicted as hyperintense relative to surrounding tissues, as demonstrated in FIG. 1D (arrow). Thrombus signal/noise ratio was 153±57 and 261±95 for group 1 (n=10) and group 2 (n=13), respectively. Thrombus-to-lumen CNR was 149±57 and 256±94 for group 1 and group 2. Thrombus to brain tissue CNR was 41±36 and 120±63 (P<0.01), respectively. The difference between the 2 groups were significant in all above signal measurements (FIG. 2).

### Diagnostic Performance of MRBTI

MRBTI correctly identified 113 of 116 segments with CVT with a sensitivity of 97.4%. In 527 of 531 segments, CVT was ruled out correctly with a specificity of 99.3%. A detailed overview of the diagnostic performance of MRDTI compared with standard of reference is summarized in Table 2. MRBTI was able to detect hyperintense thrombi in different segments.

TABLE 2
Diagnostic Performance of MRBTI for Detection of CVT

| | Patient Based | | Segment Based | |
|---|---|---|---|---|
| | n = 47 | | n = 647 | |
| CVT by consensus reading, n | 23 | | 116 | |
| CVT by MRBTI, n | 23 | | 113 | |
| False positive, n | 1 | | 4 | |
| False negative, n | 0 | | 3 | |
| Sensitivity, % (95% CI) | 100 | (85.2-100) | 97.4 | (92.6-99.5) |
| Specificity, % (95% CI) | 95.8 | (78.9-99.9) | 99.25 | (98.1-99.8) |
| Positive predictive | 95.8 | (78.9-99.9) | 96.6 | (91.5-99.1) |
| value, % (95% CI) | | | | |
| Negative predictive | 100 | (85.2-100) | 99.4 | (98.4-99.9) |
| value, % (95% CI) | | | | |

Data are presented as percentages, with raw data in parentheses and 95% CI.
CI indicates confidence interval; CVT, cerebral venous thrombosis; and MRBTI, magnetic resonance black-blood thrombus imaging

### Quantification of Thrombus Volume

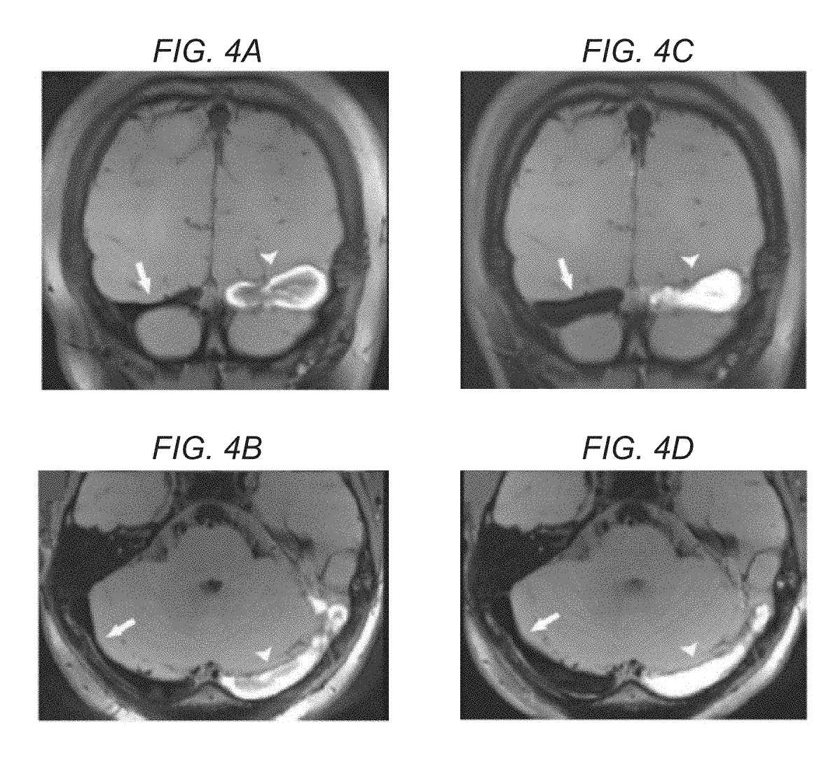

Quantification of thrombus volume was successfully conducted in all patients with CVT. Mean volume of thrombus was 10.5±6.9 mL. There was no significant difference between the 2 groups (8.6±7.2 versus 11.9±6.5 mL; P=0.28). FIG. 4 demonstrates thrombus volume quantification in 1 patient who underwent series scans on day 7 and day 14 (22.4 versus 12.5 mL). The complicated signal intensity pattern of evolving thrombus was also revealed.

### Discussion

The results described above demonstrated that MRBTI can detect CVT early with a high diagnostic accuracy. This study is believed to be the first evaluation of MRBTI for direct visualization of CVT.

Neuroimaging plays a key role in the diagnosis of CVT. CT venography and MRV have been widely used for detecting cerebral venous changes that may be related to thrombosis. Instead of directly imaging thrombus, most of these techniques rely on visualization of altered blood flow in the veins resulting from thrombotic vessel lumen. Anatomic variants of normal venous anatomy, including sinus atresia/hypoplasia, asymmetrical sinus drainage, and normal sinus filling defects, may mimic sinus thrombosis and compromise the diagnostic confidence using these methods. For example, arachnoid granulations protruding into the sinus lumen may produce a focal filling defect on MRV that can simulate focal thrombosis. Contrast-enhanced MRV with elliptic centric ordering has been widely used as a venographic method, which may assist in distinguishing anatomic variants from CVT. However, it has limited utility in patients with renal impairment because of the requirement of gadolinium.

Unlike conventional imaging techniques, MRBTI directly targets the thrombus itself and depicts thrombus as hyperintense and other tissues as isointense based on strong T1 contrast weighting. Despite the sufficient contrast for thrombus detection with conventional thrombus imaging technique, the volume of thrombus could be underestimated due to sometimes heterogeneous appearance in acute or subacute thrombus. To overcome the limitation, T1-weighted variable flip angle turbo spin echo is applied to CVT detection by using its intrinsic blood nulling capability. The results discussed above demonstrated that CVT was well isolated from the surrounding tissues, including lumen and wall with this MRBTI method, and the entire thrombus volume is readily appreciated. In addition, sinus anatomy structures, such as sinus wall, arachnoid granulations, and surrounding tissues, can be well visualized. On the other hand, the black-blood contrast helps reduce the false-positive diagnosis because of flow artifacts commonly observed on time-of-flight, as shown in FIG. 1. This suggests that the black-blood feature was a major contributor to the high detection accuracy of CVT in this study.

The feasibility of quantifying thrombus volume was demonstrated as well. Because of the high signal contrast of thrombus and clear spatial depiction of venous structure as mentioned above, the volume of CVT can be quantified with the aid of software in a semiautomatic fashion. Such a quantitative method makes MRBTI a robust technique for monitoring thrombus progression.

The noncontrast nature of MRBTI is highly relevant to clinics. The technique is free of the risk for allergic reactions and can be used for repeated follow-up examination if necessary. A substantial patient population, including pregnant and postpartum women and the elderly with severe kidney insufficiency, will greatly benefit from such a radiation free, noncontrast imaging technique. Advantageously, the MRBTI technique can serve as a first-line diagnostic imaging test.

Of note, hyperacute or chronic thrombosis does not have a short T1 relaxation time and exhibits isointense signal on T1-weighted images. However, with the blood signal adequately suppressed on MRBTI, normal venous anatomy was depicted as hypointense black area. Therefore, thrombus with isointense signal can still be readily detected. In addition, the presence of high signal intensity in chronic CVT patients could be used as a conclusive sign of a recurrent CVT. It is also worth noting that by using the highly efficient variable flip angle turbo spin echo technique described above for data acquisition, the imaging time of the current protocol is about 6-8 minutes, however, further acceleration of data acquisition (such as elliptical acquisition, advanced parallel imaging, and compressed sensing) would be possible. Finally, the reference gold standard used in this study was a combination of conventional imaging techniques (CT, MR, and MRV) with clinical information.

In conclusion, the findings described above support that in various embodiments, MRBTI allows selective visualization of thrombus with high accuracy and provides a valuable alternative to current techniques, opening a new place for MRBTI in CVT diagnostics.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method for performing magnetic resonance imaging (MRI) on a subject, comprising (1) acquiring magnetic resonance data from a volume of interest (VOI) comprising a blood vessel of a subject's head and/or neck, by using an MRI machine to perform a 3D variable-flip-angle turbo spin-echo (TSE) acquisition, and (2) generating one or more images based on said data.

2. The method of claim 1, wherein the blood vessel comprises a thrombus.

3. The method of claim 2, wherein the thrombus is depicted as hyperintense compared with surrounding tissues.

4. The method of claim 1, wherein the subject has cerebral venous thrombosis (CVT).

5. The method of claim 1, further comprising using T1-weighting.

6. The method of claim 1, wherein the MRI machine is a 1.5 T scanner or a 3.0 T scanner.

7. The method of claim 1, wherein the VOI comprises one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé , right cortical veins, and left cortical veins.

8. The method of claim 1, wherein the subject is a human.

9. A magnetic resonance imaging (MRI) system, comprising:
(1) a magnet operable to provide a magnetic field;
(2) a transmitter operable to transmit to a region within the magnetic field;
(3) a receiver operable to receive a magnetic resonance signal from the region; and
(4) a processor operable to control the transmitter and the receiver; wherein the processor is configured to direct the transmitter and receiver to execute a sequence, comprising (a) acquiring magnetic resonance data from a blood vessel within a volume of interest (VOI) that comprises all or a portion of a subject's head and/or neck, according to the method of claim 1, and (b) generating one or more images based on the magnetic resonance data acquired.

10. The MRI system of claim 9, wherein the system comprises a head and/or neck coil.

11. The MRI system of claim 9, wherein the system is configured to image one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins.

12. The MRI system of claim 9, wherein the subject is a human.

13. The MRI system of claim 9, further comprising a subsystem configured to accelerate imaging speed via parallel processing.

14. The MRI system of claim 9, wherein the MRI system is a 1.5 T system or a 3.0 T system.

15. A non-transitory machine-readable medium having machine executable instructions for causing one or more processors of a magnetic resonance imaging (MRI) machine, and/or a subsystem configured to function therewith, to execute an imaging method, said method comprising: performing a 3D variable-flip-angle turbo spin-echo (TSE) acquisition of a blood vessel within a volume of interest (VOI) comprising a subject's head and/or neck.

16. The non-transitory machine-readable medium of claim 15, wherein the imaging comprises T1-weighting.

17. The non-transitory machine-readable medium of claim 15, wherein the MRI machine comprises a head and/or neck coil.

18. The non-transitory machine-readable medium of claim 15, wherein the VOI comprises one or more of the following anatomical structures or regions within the subject: superior sagittal sinus, right transverse sinus, right sigmoid sinus, left transverse sinus, left sigmoid sinus, straight sinus, confluence of sinuses, veins of galen, internal cerebral veins, veins of Labbé, right cortical veins, and left cortical veins.

19. The non-transitory machine-readable medium of claim 15, wherein the blood vessel comprises a thrombus.

20. The non-transitory machine readable medium of claim 15, wherein subject is a human.
